(19) United States
(12) Patent Application Publication
(10) Pub. No.: US 2004/0133136 A1
(43) Pub. Date: Jul. 8, 2004

(54) Method and film for fixing an equipment, in particular for medical use, on an external part of a living body, and equipment provided with such a film

(76) Inventors: Paul Lassalle, Biarritz (FR); Florence Roux, Biarritz (FR); Francois Capitaine, Anglet (FR)

Correspondence Address: MICHAEL BEST & FRIEDRICH, LLP, 100 E WISCONSIN AVENUE, MILWAUKEE, WI, 53202, US

(21) Appl. No.: 10/478,586

(22) PCT Filed: May 24, 2002
(86) PCT No.: PCT/FR02/01752

(30) Foreign Application Priority Data
May 28, 2001 (FR) 01/06918

(51) Int. Cl. A61F 5/00

(52) U.S. Cl. 602/3

(57) ABSTRACT

The invention consists in a film for fixing an appliance, in particular for medical use, on an external portion of a living body, the film being coated on a first face in an adhesive layer for fixing it to the external portion, and being fixed on its other face to the appliance. In the invention, said first face presents recesses regularly distributed thereover so as to constitute a locally-discontinuous adhesion interface between the film and the adhesive layer. The invention is particularly applicable to fixing a bag for collecting physiological effluent to the abdomen of a patient who has been subjected to a stoma operation.

The invention relates to a method and to a film for fixing an appliance, in particular for medical use, on an external portion of a living body of a human or an animal, and in particular for fixing a bag for collecting physiological effluent onto the abdomen of a patient who has been subjected to a stoma operation. The invention also relates to an appliance for medical use and fitted with such a film.

### BACKGROUND OF THE INVENTION

It is common to need to fix appliances, generally for medical use, and generally on a temporary basis, to an external portion of the body of a patient, for example during a surgical operation or after an operation. Mention can be made in particular of fixing the needle of a drip feed device to the arm of a patient, putting a patch into place, e.g. for measuring heart rate, or for administering medical or other treatment, or indeed for fixing appliances of greater volume such as a bag for collecting physiological effluent that is put into place on a patient who has been subjected to a stoma operation.

In order to fix such appliances, it is common practice to use films of plastics material or of fabric which are coated in an adhesive layer such as a glue or a gum for adhering to the skin, and which are also fixed to the appliance, generally via a portion of their area. They can comprise an adhesive tape like a strip of plaster dressing, an adhesive plate, or a patch. The adhesive layer used generally presents good adhesion characteristics on the skin of the patient, and it is preferably hypoallergenic.

It has been found that such adhesive tapes or patches do not present satisfactory adhesion characteristics over time, in particular at the interface between the adhesive layer and the film supporting said layer. The film becomes detached from the adhesive layer and total separation can happen, particularly if the appliance is bulky or heavy, such as a stoma bag, or if the film is likely to be moistened by body fluids or medical substances. This can thus be particularly inconvenient for the patient since the appliance might then no longer function correctly.

### OBJECT AND SUMMARY OF THE INVENTION

The object of the invention is to provide good adhesion in order to avoid appliances becoming detached.

For this purpose, the invention provides for the adhesion between the film and the adhesive layer to be locally discontinuous and regularly distributed.

More precisely, the invention provides a method of fixing an appliance on an external portion of a living body, in which a locally-discontinuous adhesion interface is created between a face for connection to the appliance and an adhesive layer for connection to the external portion by means of a regular distribution of interruptions of contact formed on the face, with the adhesive layer penetrating at least in part via said interruptions.

The invention also provides a fixing film for fixing an appliance, in particular for medical use, on an external portion of a living body, the film being coated on a first face in an adhesive layer for fixing to the external portion and being fixed, on its other face, to the appliance, in which said first face presents recesses that are regularly distributed thereover in order to constitute a locally-discontinuous adhesion interface between the film and the adhesive layer, and the adhesive layer penetrates at least in part into at least some of the recesses in the film.

In an embodiment, the recesses are in the form of orifices passing through the thickness of the film, each orifice having a wall opening out in diverging manner into said first face of said film and in converging manner into the other face of the film so that the film presents a cellular type structure.

Furthermore, the films used in the prior art are generally lacking in elasticity and porosity. They are therefore sometimes poorly adapted to the zones of the patient's body onto which they are fixed, and their stiffness then contributes to their becoming detached.

Preferably, in accordance with the invention, the film is a flexible film, in particular made of polyolefins or of non-woven fibrous material, for example made up of fine polyester fibers, and the adhesive layer is an elastomer gum, for example based on isobutylene and sodium carboxymethylcellulose.

According to an additional aspect, the film presents an elastic range stretch ratio that is large, possibly being as great as about 300%.

Advantageously, a removable protective sheet covers the adhesive layer prior to said film being used.

The invention also provides an appliance for medical use provided with a film as described above to enable the appliance to be fixed on an external portion of a living body.

In particular, the appliance may be a bag for collecting physiological effluent for fixing by means of said flexible film to the abdomen of a patient who has been subjected to a stoma operation.

Advantageously, the film is fixed to the bag by means of an annular line of heat-sealing and the bag is stuck on the patient's abdomen via an adhesive layer.

### BRIEF DESCRIPTION OF THE DRAWINGS

Other characteristics, details, and advantages of the invention appear on reading the following description made with reference to the accompanying drawings given by way of example and in which:

FIG. 1 is a section view of a cellular film provided with an adhesive layer on one of its faces;

FIG. 2 is a cutaway perspective view of FIG. 1;

FIG. 3 is a section view showing a variant of the FIG. 1 embodiment in which the adhesive layer penetrates part of the way into the recesses formed in the thickness of the film; and

FIG. 4 shows a portion of a bag for collecting physiological effluent from a patient who has been subjected to a stoma operation, said bag being fitted with a film of the invention.

### MORE DETAILED DESCRIPTION

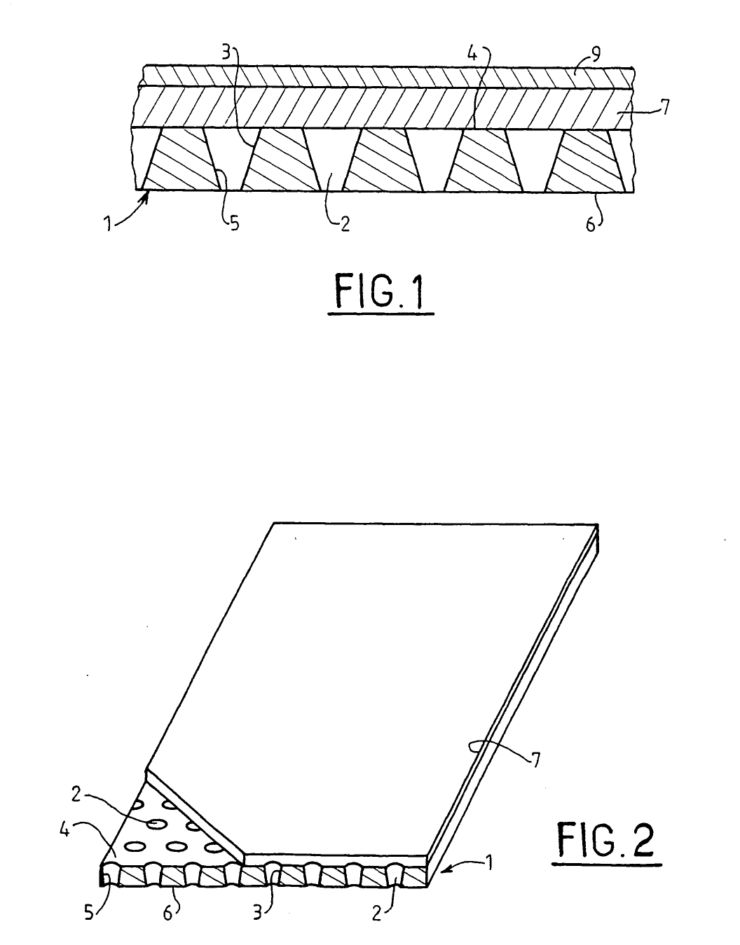

A film 1 in accordance with the invention is shown in section in FIG. 1. In this embodiment, the flexible film 1 is made of a synthetic material based on a polyolefin and presents thickness E of less than 1 millimeter. The film 1 is provided with a plurality of recesses in the form of orifices 2 passing through its thickness E in such a manner as to give said film 1 a cellular structure. Each orifice 2 is defined by a wall 3 which opens out in diverging manner towards a first face 4 of the film 1 and a wall 5 which extends the wall 3 and which opens out in converging manner in a second face 6 of the film 1 opposite from the first face 4. Each orifice is thus in the form of a truncated circular cone.

As can also be seen in FIG. 2, a layer of adhesive 7, e.g. an elastomer gum based on isobutylene and on sodium carboxymethylcellulose covers the connection face 4 into which the diverging walls 3 of the orifices 2 in the film 1 open out.

Because of the presence of the orifices 2 which are preferably distributed in regular manner in the thickness of the film 1, the connection face 4 thus constitutes a locally-discontinuous adhesion interface between the adhesive layer 7 and the film 1. This discontinuity of contact between the film and the adhesive layer at said orifices 2 improves the adhesion of the adhesive layer on the film.

In a first embodiment shown in FIGS. 1 and 2, the adhesive layer 7 merely covers the orifices 2.

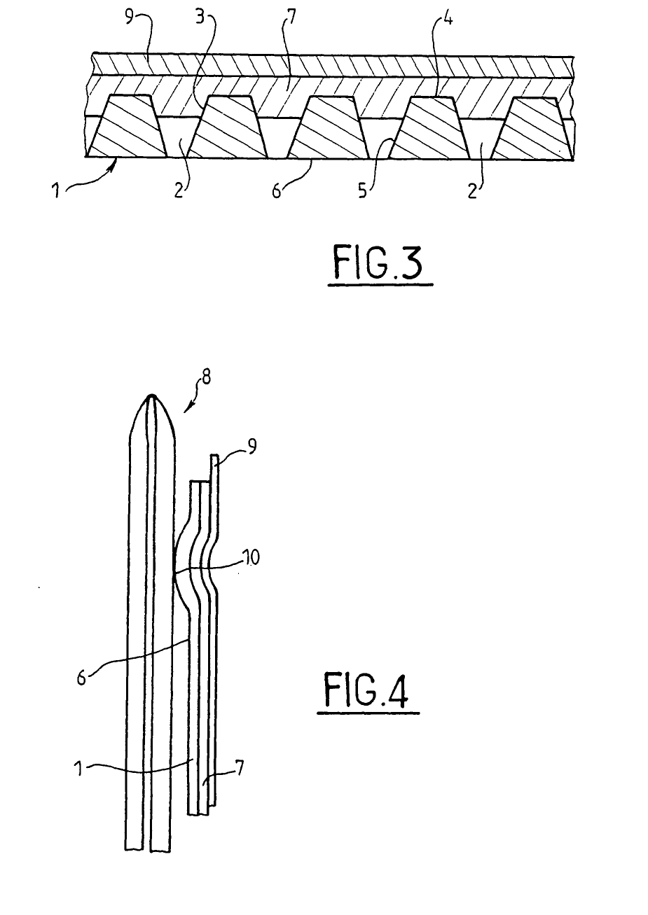

In a second embodiment shown in FIG. 3, the adhesive layer 7 penetrates at least part of the way into the orifices 2 so as to hold the film 1 more strongly by means of better cohesion therewith.

A particular application of this film is shown in FIG. 4. After a surgical stoma operation performed due to a malformation, a tumor, or an inflammation, and in particular after a colostomy, an ileostomy, or a urostomy, it is necessary to reestablish the digestive and/or urinary functions of the operated patient and mitigate the partial or total ablation of certain organs including in particular the colon, the small intestine, the rectum, and the bladder. For this purpose, the stomized patient is generally provided with a bag that is put into place immediately after the operation around an abdominal orifice for discharging physiological effluent.

A bag 8 for collecting physiological effluent, such as urine or human or animal feces, is thus shown in FIG. 4. This bag 8 is provided with a flexible film 1 enabling it to be fixed to the abdomen of the patient, the adhesive layer 7 of the film being covered by a removable protective sheet 9 prior to the film 1 being used. The film 1 is secured to the bag 8 via its face 6 opposite from the adhesive layer 7, e.g. by means of an annular line of heat-sealing 10, as shown in FIG. 4.

The cellular and extremely flexible nature of the film, and its great ability to take up the shape of the patient's skin contribute to the comfort and the discreteness with which the bag 8 can be worn. The assembly comprising the film 1 and the adhesive layer 7 presents, in particular, an elastic range stretch ratio that is large, possibly being as much as about 300%, with it being capable of returning to its initial state. In addition, flexible behavior while in traction makes it possible to conserve the continuity of the adhesive layer 7, i.e. without rupture, even for large amounts of stretching.

Furthermore, since the film 1 is stuck to the patient's skin by means of a layer 7 of an adhesive that is suitable for this purpose, the cellular nature of the film 1 and the converging-diverging shape of the orifices 2 serve to improve the cohesion or indeed the anchoring of the adhesive layer 7 with the film 1, particularly when this cohesion is subjected to the potential hazard of traces of physiological liquid infiltrating after seeping out from the surroundings of the stoma.

Nevertheless, it must be understood that these examples are given purely in order to illustrate the subject matter of the invention and they do not constitute any kind of limitation thereon.

Thus, the invention is not limited to fixing an appliance for medical use on a human being, but can also be used on an animal.

The recesses are not necessarily orifices that pass through the thickness of the film. Thus, it is possible to have recesses that are oblong, or even longitudinal channels possibly crossing one another in order to form a grid. The purpose is to provide a locally-discontinuous adhesion interface between the film and the layer of adhesive in order to improve cohesion between them.

In the particular case of orifices passing through the thickness of the film, these orifices may be of various shapes other than frustoconical. For example, it is possible to envisage an hourglass shape or indeed a circularly cylindrical shape or a prismatic shape on a square or rectangular base.

The adhesive layer may naturally have a chemical composition other than that given by way of example providing it ensures good adhesion both on a living body, and in particular on the skin of a human being, and on the film, while preferably also being hypoallergenic and relatively elastic.

The same applies to the film which may be made of a material other than a polyolefin.

What is claimed is:

1. A method of fixing an appliance on an external portion of a living body, wherein a locally-discontinuous adhesion interface is created between a face for connection to the appliance and an adhesive layer for connection to the external portion by means of a regular distribution of interruptions of contact formed on the face, with the adhesive layer penetrating at least in part via said interruptions.

2. A fixing film for fixing an appliance, in particular for medical use, on an external portion of a living body, the film being coated on a first face in an adhesive layer for fixing to the external portion and being fixed, on its other face, to the appliance, wherein said first face presents recesses that are regularly distributed thereover in order to constitute a locally-discontinuous adhesion interface between the film and the adhesive layer, and wherein the adhesive layer penetrates at least in part into at least some of the recesses in the film.

3. A fixing film according to claim 2, in which the recesses are in the form of orifices passing through the thickness of the film, each orifice having a wall opening out in diverging manner into said first face of said film and in converging manner into the other face of the film so that the film presents a cellular type structure.

4. A fixing film according to claim 2, the film being made of flexible material.

5. A fixing film according to claim 2, the film being made of polyolefins.

6. A fixing film according to claim 2, the film being constituted by a non-woven fiber fabric.

7. A fixing film according to claim 6, the film being made of polyester fibers.

8. A fixing film according to claim 2, in which the adhesive layer is an elastomer gum.

9. A fixing film according to claim 8, in which the elastomer gum is based on isobutylene and on sodium carboxymethylcellulose.

10. A fixing film according to claim 2, in which a removable protective sheet covers the adhesive layer prior to use of said film.

11. A fixing film according to claim 2, in which said film presents an elastic range stretch ratio of up to about 300%.

12. An appliance for medical use, the applicance being provided with a flexible film according to claim 2 to enable it to be fixed on an external portion of a living body.

13. An appliance according to claim 12, in which said appliance is a bag for collecting physiological effluent and for fixing by means of said flexible film to the abdomen of a patient who has been subjected to a stoma operation.

14. An appliance according to claim 13, in which the flexible film is fixed to the bag by means of an annular line of heat-sealing, and the bag is stuck to the patient's abdomen via the adhesive layer.
